(19) United States
(12) Patent Application Publication
(10) Pub. No.: US 2010/0216247 A1
(43) Pub. Date: Aug. 26, 2010

(54) Identification of Biomarkers for Screening and Monitoring of Prostate Cancer and Prostate Related Diseases

(75) Inventors: Arijit Bose, Lexington, MA (US); Nazneen Aziz, Lexington, MA (US); Ashish Jha, Berkeley, CA (US)

Correspondence Address: GREENBERG TRAURIG, LLP, ONE INTERNATIONAL PLACE, 20th FL, ATTN: PATENT ADMINISTRATOR, BOSTON, MA, 02110, US

(73) Assignee: Vitrimark, Inc.

(21) Appl. No.: 12/622,291

(22) Filed: Nov. 19, 2009

Related U.S. Application Data
(60) Provisional application No. 61/116,054, filed on Nov. 19, 2008.

(51) Int. Cl. G01N 33/48

(52) U.S. Cl. 436/63

(57) ABSTRACT

A system and method for determining presence of prostate cancer or prostate related diseases is provided. The method includes first obtaining a test sample from a test subject. Next, at least one ultrastructural biomarker indicative of prostate cancer within the sample may be identified. Thereafter, qualitative or quantitative data from the ultrastructural biomarker in the test sample can be obtained. The qualitative or quantitative data of the biomarker in the sample from the test subject can then be compared to that in a sample from a control subject for variations. The presence of qualitative and quantitative variations can act as a determinant for prostate cancer or prostate related diseases.

### RELATED U.S. APPLICATIONS

The present application claims priority to U.S. Provisional Application Ser. No. 61/116,054 filed Nov. 19, 2008, which application is hereby incorporated herein by reference.

### TECHNICAL FIELD

This invention relates generally to imaging systems and methods, and more particularly to imaging systems and methods for identifying ultrastructural biomarkers for subsequent screening and monitoring of prostate cancer or prostate related diseases in humans and animals.

### BACKGROUND ART

The deepening productivity crisis in the pharmaceutical industry, the high cost to the pharmaceutical industry of introducing new drugs to the market, partly because of expenses related to Phase I, II and III clinical trials, as well as late stage failures for many drug candidates have spurred intense across-the-board activity around biomarker discovery and validation. Biomarkers, defined by the FDA as a characteristic that can be objectively measured and evaluated as an indicator of normal biologic or pathogenic processes or pharmacological responses to a therapeutic intervention, are being sought actively to help make early and cost-effective “go/no-go” decisions on drugs, for patient stratification, clinical trial analysis, and finding niche markets (e.g., sub-population of patients who respond to drugs or in whom no drug-related toxicity can be seen) for new drugs under development. In addition, the FDA has recently recommended that validated or investigational biomarker data be included in IND and NDA packages. These are powerful drivers for the biomarker market, whose size can be estimated at about $428 millions in 2005, and has since been growing at 20 percent per year.

The use of biomarkers is rapidly gaining momentum in the pharmaceutical industry and in the medical management of patients. Current methods for identifying biomarkers involve the use of biochemical assays for identifying “functional” biomarkers, such as genes or protein arrays or metabolite analysis. The use of biochemical assays in this context requires probing for functional alterations in genes and proteins, the need for a priori knowledge of their function, as well as extensive assay development and optimization.

While there has been an explosion of biomarker discovery efforts utilizing genomics, proteomics and metabolomics, these technologies also focus only on functional biomarkers. With many diseases, the presence of observable functional biomarkers often occurs late in the disease state. As such, preventive measures for these diseases may be ineffective when developed in connection with the management of the disease, or in early evaluation of drug efficacy.

Contributions towards understanding ultrastructural morphology have been made in recent years. Such an approach focuses on the ultrastructural differences in the biological samples that can occur much earlier in the diseased state, even before functional differences are observable. Since these target structures typically range from between about 5 nanometers (nm) and 1 micrometer, one approach to visualizing them is through the use of conventional transmission electron microscopy (TEM). However, the use of conventional TEM has some critical limitations. For example (i) the high vacuum used in TEM removes solvent, leaving behind structures that are quite different from those present in the original solution, (ii) adequate contrast between the sample features and background is usually not available, necessitating the use of stains (the addition of stains, which usually are heavy metal salts, however can cause dramatic changes in the morphology of the structures), and (iii) the exposure of the sample to the electron beam often damages the sample.

Understanding the ultrastructural morphology of a disease like prostate cancer, for instance, may provide insight in detecting and monitoring the progression of the disease. Prostate cancer afflicts a very significant fraction of the aging male population with approximately 200,000 new cases diagnosed each year in the US. Prostate cancer varies in stage and grade, and it is this stage and grade that determines the prognosis and possible treatment options. The stage and grade of prostate cancer can be classified using a Gleason score. Gleason scores typically range from 2 to 10. Most prostate cancer cases diagnosed have Gleason scores of 5, 6 or 7. The more aggressive forms of prostate cancer have scores of 8, 9 or 10. Gleason scores below 4 are rare since they usually do not warrant the biopsy in the first place.

For prostate cancer, prostate specific antigen (PSA) has been commonly used as a diagnostic biomarker. Levels of PSA below 4 ng/ml in blood are considered normal, whereas levels of PSA between 4 ng/ml and 10 ng/ml are in the borderline or grey zone, while levels of PSA greater than 10 ng/ml are a strong indicator for the presence of the disease. While this test remains in widespread use today, it can lead to both false negative as well as false positive indications. For example, only about 25-30% of men who have a biopsy due to elevated PSA levels actually have prostate cancer. The normally slow-growing nature of prostate cancer often means that cancer can be present even if the PSA level is low. Alternate strategies include monitoring of hypermethylation of the glutathione S-transferase P1 (GSTP1) promoter. Hypermethylation of this promoter can inactivate the tumor suppressor function of this gene. However, the inactivation of this promoter has not been linked causatively to the disease. Other structural biomarkers that have been used are serum osteoprotegerin levels, human tissue kallikreins, changes in serum levels of sICAM-1 and sVCAM-1, e-cadherin fragments, serum amyloid A and serum caveolin-1. To date, there may not be a clear structural biomarker that represents predisposition, onset or progression of the disease.

Accordingly, it would be desirable to identify substantially more sensitive and specific biomarkers that can be used in the screening and monitoring of prostate cancer, and in monitoring the efficacy and toxicity of drugs for treatment.

### SUMMARY OF THE INVENTION

The present invention provides, in one embodiment, a method for determining presence of prostate cancer or prostate related diseases. The method includes first obtaining a test sample from a test subject. Next, at least one ultrastructural biomarker indicative of prostate cancer within the sample may be identified. Thereafter, qualitative or quantitative data from the ultrastructural biomarker in the test sample can be obtained. The qualitative or quantitative data of the biomarker in the sample from the test subject can then be compared to that in a sample from a control subject for variations. The presence of qualitative or quantitative variations can act as a determinant for prostate cancer or prostate related diseases.

In accordance with another embodiment of the present invention, a method for screening, monitoring, predicting or assessing susceptibility to prostate cancer or prostate related diseases is provided. The method includes initially obtaining a first sample from a subject. Next, at least one ultrastructural biomarker indicative of prostate cancer within the first sample may be identified. Thereafter, qualitative or quantitative data from the ultrastructural biomarker in the first sample can be obtained. The qualitative or quantitative data of the biomarker in the first sample from the subject can then be compared to that in a second sample from the subject for variations. The presence of qualitative or quantitative variations can be associated with a predictor for prostate cancer or prostate related diseases.

In accordance with a further embodiment of the present invention, a method for evaluating or predicting therapeutic efficacy or response is provided. The method includes initially obtaining a test sample from a test subject. The method includes initially obtaining a first sample from a subject. Next, at least one ultrastructural biomarker indicative of prostate cancer within the first sample may be identified. Thereafter, qualitative or quantitative data from the ultrastructural biomarker in the first sample can be obtained. The qualitative or quantitative data of the biomarker in the first sample from the subject can then be compared to that in a second sample from the subject for variations. The presence of quantitative or qualitative variations can be equated with a marker of therapeutic efficacy.

In accordance with another embodiment of the present invention, a method for assessing risks of toxicity, adverse events, serious adverse events associated with a drug or therapeutic candidate for treatment of prostate cancer or other prostate related disease is provided. The method includes initially obtaining a first sample from a subject. Next, at least one ultrastructural biomarker indicative of prostate cancer within the first sample may be identified. Thereafter, qualitative or quantitative data from the ultrastructural biomarker in the first sample can be obtained. The qualitative or quantitative data of the biomarker in the first sample from the subject can then be compared to that in a second sample from the subject for variations. The presence of qualitative or quantitative variations can be correlated as a marker for risks for toxicity, adverse events or serious adverse events associated with the drug or therapeutic candidate.

### BRIEF DESCRIPTION OF DRAWINGS

FIGS. 1A-C illustrate a system for use in preparing samples for subsequent imaging and identification of ultrastructural biomarkers, in accordance with an embodiment of the present invention.

FIG. 2 illustrates a cryotransfer station onto which a sample may be transferred for subsequent placement into a high contrast imaging device for imaging and identifying ultrastructural biomarkers, in accordance with an embodiment of the present invention.

FIG. 3 illustrates schematically another method for preparing samples for subsequent imaging and identification of ultrastructural biomarkers, in accordance with an embodiment of the present invention.

FIGS. 4A-E illustrate various embodiments of ultrastructural prostate cancer biomarkers, in accordance with one embodiment of the present invention.

FIGS. 5A-F illustrate cryo-TEM images of a human blood serum sample, in accordance with an embodiment of the present invention.

FIGS. 6A-B illustrate cryo-TEM images of a human blood serum sample, in accordance with an embodiment of the present invention.

FIGS. 7A-C illustrate cryo-TEM images of a murine blood serum sample, in accordance with an embodiment of the present invention.

FIGS. 8A-F illustrate a change comparison in qualitative and quantitative data between a control sample and a diseased sample, in accordance with an embodiment of the present invention.

### DESCRIPTION OF SPECIFIC EMBODIMENTS

In accordance with one embodiment of the present invention, systems and methods are provided to monitor serum-based structural biomarkers for prostate cancer and also to identify a more sensitive and specific biomarker for determining predisposition, onset and progression of prostate cancer and prostate related diseases. Using image analysis, gross as well as subtle differences in nanoscale structures in the serum between healthy individuals and prostate cancer patients may be obtained. It should be appreciated that the term “nano”, “nanoscale” or the like, used in connection with the present invention, means having characteristics or features on a scale of from about 1 nanometer to about 100 nanometers, as defined by the terminology standards set forth by ASTM International. In addition, it should also be appreciated that the terms “nanoscale structures”, “ultrastructural biomarkers” and “nanostructure” may be used interchangeably throughout the application.

In an embodiment, a method of comparing profiles of the ultrastructural biomarkers uses a “difference mapping” or “finger printing” approach and may be similar to the approach used in metabolomics, where differences in the locations or intensities of metabolite peaks are compared without any knowledge of the nature of the metabolite. In addition, the method of the present invention may use, in an embodiment, a global analysis to map differences in the nanostructures (i.e., ultrastructural biomarkers) present in serum from a prostate cancer subject as compared to serum from a healthy subject. These disease specific ultrastructural biomarkers may be identified and validated by their presence and/or absence or by changes in their shape, size or other morphologies, that can be correlated consistently and reproducibly with prostate cancer. Similarly, the method of the present invention can be applied to other diseases, including other prostate related diseases, such as enlarged prostate, benign prostate hyperplasia or prostatitis. Each of these different diseases can have a distinct profile or similar profile.

In accordance with an embodiment of the present invention, uncovering differences in the morphologies or the presence/absence of a specific nanostructure in biological fluids of disease samples can provide an innovative approach to biomarker discovery. These physical ultrastructural biomarkers may not give information about the cause or etiology of the disease, but can serve as a correlative to the presence or potentially the progression of disease. In addition, when utilized in a drug development process, this high resolution detection of structural biomarkers observed in body fluids of clinical trial subjects may potentially be able to detect early signs of adverse events or efficacy.

It is important to note that the method of the present invention can be applied not only to identify what changes are occurring in the profiles of the biomarkers, but can also be employed in connection with a “difference mapping” approach to determine what changes can be indicative of prostate cancer or prostate related diseases. As such, the method of the present invention can alleviate reliance on biochemical assays which probe functional alterations in genes and proteins, need a priori knowledge of their function, and require extensive assay development and optimization. In accordance with an embodiment of the present invention, the method may allow probing functional alterations in genes and proteins by cryoimaging without bias and also patterning differences as the diagnostics or biomarker.

In addition, it should be appreciated that different types of ultrastructural changes in connection with ultrastructural biomarkers may occur in prostate cancer or prostate related diseases. In one embodiment, these ultrastructural changes may include primary changes and/or secondary changes. In accordance with an embodiment, primary changes may include changes in protein primary sequence due to amino acid alterations (single base mutations in the gene) in cell cycle regulatory genes, which may result in secondary or tertiary structure modifications. These ultrastructural changes in the primary target gene (oncogene or tumor suppressor gene) can lead to abnormal levels of expression or altered post-translational modifications (phosphorylation/glycosylation/acetylation) in downstream pathway proteins, such as, within intracellular signaling proteins or in the tumor microenvironment. Within the tumor microenvironment, increased necrosis, angiogenesis, or changes in the reactive stroma or extracellular matrix may occur.

In addition to primary changes, secondary changes may also occur within the ultrastructural biomarkers. The secondary abnormalities may include protein level changes, abnormal protein-protein interactions or post-translational modifications. Therefore, besides the expected changes in morphology within the primary mutated protein target, there may be alterations in morphologies of many other protein-complexes. Shape and structural variations may also occur from alterations in the interactions of protein-protein, protein-lipid or protein-glycoprotein that occur due to perturbations in the level of one or more components of these macromolecular complexes in the course of the disease. Using this insight, relative spatial positions of proteins in a larger assembly can also be identified. This is important because proper assembly can be critical to the functioning of protein complexes and cell organelles. Because these changes are physical, the identification process does not require any a priori knowledge of specific biological targets.

Ultrastructural changes, however, may not be limited to protein complexes within the secreted components. In an embodiment, ultrastructural alterations may also occur within lipid vesicles, carbohydrate, lipoproteins, glycoproteins, RNA/DNA complexes, or metabolite complexes. Any structural changes within these complexes or presence or absence of any of these components can also be reflected as an alteration in the profile of nanostructures in the serum. In prostate cancer, for instance, there are lipid metabolism abnormalities, such as increased requirement of LDL cholesterol, fatty acids and prostaglandin synthesis which may not be found in the normal prostate tissue.

In accordance with an embodiment of the present invention, uncovering differences in the morphologies or the presence/absence of an ultrastructural biomarker, in biological fluids of disease samples provides an innovative approach to biomarker discovery, and the presence/absence of these ultrastructural biomarkers may serve as in indicator or predictor of the presence or potentially the progression of disease. In an embodiment, the method of the present invention may use a highly sensitive artifact-free imaging technique to reveal morphological details of ultrastructural biomarkers. For instance, imaging techniques that may be used include cryogenic transmission electron microscopy, or Cryo-TEM as set forth in U.S. Pat. No. 7,507,533, which is hereby incorporated by reference. In an embodiment, imaging techniques may include modified freeze fracture direct imaging (M-FFDI). Other highly sensitive imaging techniques, for instance, near field scanning optical microscopy (NSOM), may be used as well to identify morphological differences. In addition, size measurement techniques, such as dynamic light scattering (DLS) techniques, may be used to measure the size of the ultrastructural biomarkers.

### Cryo-TEM Technique:

1. The Controlled Environmental Vitrification System

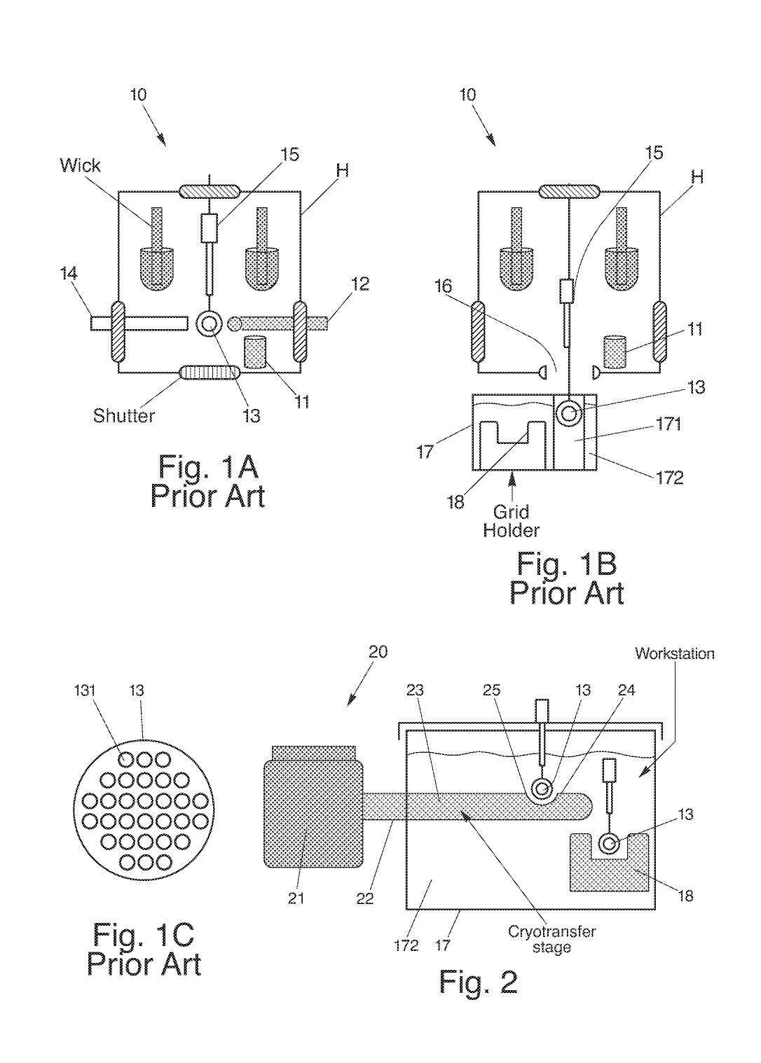

Referring now to FIGS. 1A-C, there is illustrated a Controlled Environmental Vitrification System (CEVS) 10, such as that available from The Department of Chemical Engineering & Material Sciences at the University of Minnesota, for use in the preparation of samples for subsequent ultrastructural biomarker identification. In general, the CEVS 10 includes a housing H equipped with temperature and humidity control. The CEVS 10 also includes a vial 11, within which a volume of sample may be stored. The sample within the vial 11, in one embodiment, may be thermally equilibrated relative to the interior of housing H. To extract an amount of the sample from the vial 11, a pipette 12 may be provided adjacent to the vial 11. The CEVS 10 may also include a grid 13 onto which the extracted sample may be deposited from the pipette 12. The grid 13, in one embodiment, may be a perforated disc with holes 131 that are sufficiently spaced to support the deposited sample. In an embodiment, the grid 13 may be a specially prepared holey carbon grid that is approximately 3 millimeters (mm) in diameter, if circular in shape. Of course, the grid may be designed with other geometric shapes if necessary. Such a holey carbon grid is well known in the art and includes, among other things, a perforated copper disc having a carbon layer, approximately 200 nm in thickness, extending across holes 131. The carbon layer, in an embodiment, is provided to decrease the diameter of the holes 131 on the grid 13, and does not completely cover these holes 131. In accordance with an embodiment, the holes 131 may be provided with a diameter ranging from about 1 micrometer to about 10 micrometers. In this manner, when the sample is deposited onto the grid 13, a thin film of the sample may be generated across the reduced-size holes 131. To facilitate the generation of the thin film, the CEVS 10 may be provided with a blotter 14 adjacent the grid 13 to blot excess amount of the deposited sample from the surface of the grid 13, such that an amount sufficient for generating the thin film remains on the grid 13. In addition, to maintain the grid 13 in position during the deposition process and blotting process, a plunging mechanism 15 may be provided to which the grid 13 may be affixed.

The CEVS 10 may further include a portal 16 through which the plunging mechanism 15 may extend, so as to push the grid 13 from within the CVES 10, as illustrated in FIG. 1B. In this manner the grid 13 may be immersed within a cryogenic reservoir 17 situated in proximity to the portal 16. The reservoir 17, in one embodiment, includes an inner chamber 171 within which a volume of a first cryogenic fluid may be accommodated and into which the grid 13 along with the sample may be immersed. In one embodiment, the first cryogenic fluid may be liquid ethane, or similarly cold cryogenic fluid, such as liquid propane. For ethane, the normal melting point is about −183° C., while the normal boiling point is about −89° C. The reservoir 17 may also include an outer chamber 172 situated about the inner chamber 171 for accommodating a volume of a second cryogenic fluid, such as liquid nitrogen, or a similarly cold cryogenic fluid. By positioning outer chamber 172 about inner chamber 171, the volume of liquid ethane in inner chamber 171 may be kept sufficiently cold and substantially close to its melting point by the presence of the liquid nitrogen in the outer chamber 172 to maximize heat transfer away from the sample and to allow the sample to vitrify rather than crystallize. The reservoir 17 may further include a grid holder 18 submerged within the liquid nitrogen in the outer chamber 172 for subsequent placement of the grid 13 thereon.

Although the CEVS 10 was described, it should be appreciated that other commercially available vitrification systems may also be used in the preparation of samples for subsequent ultrastructural biomarker identification. For instance, an automated system, such as the Vitrobot system from FEI Company, can be used.

2. Preparation of the Sample

Still looking at FIGS. 1A-C, approximately 1-10 microliters of the liquid sample may be withdrawn from vial 11 using pipette 12, and subsequently deposited onto perforated grid 13. In one embodiment, the sample may be about 2-5 microliters in volume, and preferably, about 3 microliters in volume. The sample may thereafter be blotted by blotter 14, so as to leave on grid 13 a thin film having a thickness ranging from about 50 nm to about 200 nm spanning the holes 131. It should be noted that thickness of the thin film may not be substantially uniform throughout. To that end, areas of the film that is relatively thin can provide an ideal location for imaging.

The sample on grid 13 may then be immersed by plunging mechanism 15 into reservoir 17, as shown in FIG. 1B, and in particular, into chamber 171 of liquid ethane having a temperature range of from about −170° C. to about −150° C. As noted above, the liquid ethane may be kept close to its melting point by the liquid nitrogen in the outer chamber 172 to maximize heat transfer from the grid. Contact of the sample on the grid 13 with the liquid ethane in chamber 171 can induce rapid vitrification of the sample on grid 13 within a few milliseconds. It should be appreciated that during vitrification, liquid, such as water, in the solution solidifies without crystallization. In this manner, substantially all of the microstructures in the sample may be preserved in their original state. The grid 13 may next be transferred from the inner chamber 171 of the reservoir 17 to the outer chamber 172 and placed onto the grid holder 18 in liquid nitrogen.

Referring now to FIG. 2, the grid 13 may thereafter be transferred from the holder 18 onto a cold stage 20 in the liquid nitrogen environment of the outer chamber 172 of reservoir 17. Transferring the grid 13 in a liquid nitrogen environment can help to maintain the integrity of the sample. As illustrated, cold stage 20 may include a container 21 within which a volume of, for instance, liquid nitrogen or any other similarly cold substance may be stored, and an arm 22 extending from the container 21. Arm 22, in an embodiment, may be designed for placement within the liquid nitrogen environment of the outer chamber 172, and may be tubular in shape. As such, arm 22 may be made from a material that can withstand immersion in liquid nitrogen. Arm 22 may also include a channel 23, so as to permit liquid nitrogen from container 21 to advance to tip 24 and maintain the temperature of the arm 22 thereat from about −170° C. to about −160° C., substantially well below the amorphous to crystalline phase transition temperature of about −155° C. in ice, to minimize any compromise to the integrity of the sample. As shown in FIG. 2, arm 22 may include a depression 25 towards tip 24 to provide an area onto which grid 13 may be placed for subsequent imaging. Cold stage 20, in one embodiment, can be a commercially available cold stage, such as the Cryotransfer System—CT3500J from Oxford Instruments or the Gatan 626DH from Gatan Inc., in Pleasanton, Calif.

Once the grid 13 has been transferred onto arm 22 of cold stage 20, the cold stage 20 may be inserted into, for instance, a high contrast imager (not shown), such as a TEM, under positive dry pressure to minimize the risks of contamination of the sample by, for example, atmospheric contaminants, including moisture. The positive dry pressure may be generated from a gas, such as nitrogen or oxygen. Alternatively, when using a cold stage, such as the Gatan 626DH, its design can allow the use of a glass tube to cover the grid arch and minimize the risks of contaminants. During imaging, such as phase contrast imaging, the tip 24 of arm 22 continues to be maintained at a temperature range well below the amorphous to crystalline phase transition temperature of about −155° C. in ice, in the electron microscope to maintain the integrity of the sample being imaged.

### M-FFDI Technique:

In samples having a viscosity that may be relatively high, i.e., greater than about 100 centipoise, for blotting to effectively thin down the samples, the use of cryo-TEM may not be sufficient. As such, the present invention contemplates the use of M-FFDI.

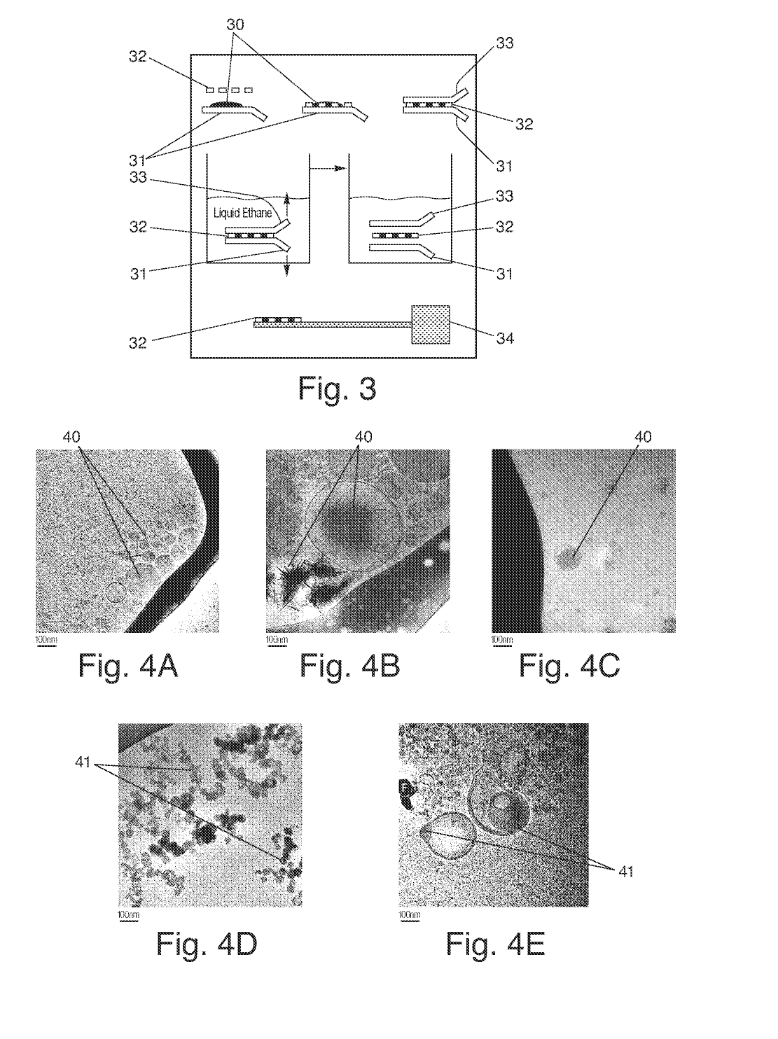

Looking now at FIG. 3, in one embodiment, approximately 100 nanoliters (nL) of the sample 30 may placed onto a plate or planchette 31. Planchette 31, in an embodiment, may be a copper planchette, or may be made from a similar material of approximately 3 mm×3 mm in size. Next, a grid 32, either standard electron microscope grid or a holey carbon grid, such as that described above, may be placed onto the planchette 31 over the sample 30, so that the sample 30 may permeate across the perforations of the grid 32. In an alternate embodiment, the sample 30 may initially be placed on the grid 32 and the grid 32 subsequently placed onto the planchette 31. The planchettes 31, in accordance with an embodiment, may be relatively larger in size than the grid 32, so as to accommodate the grid 32 thereon.

Thereafter, a second planchette 33 may be gently lowered onto the grid 32 to sandwich the grid 32 between the two planchettes 31 and 33. It should be appreciated that gentle placement of the second planchette 33 onto the grid 32 allows the sample 30 to be squeezed between the planchettes 31 and 33, and spread out over the surface of the grid 32 into previously unoccupied areas. Moreover, the spreading of the sample 30 across of the grid 32 into previously unoccupied areas generates certain thin film portions that can be substantially thinner in thickness than others across the perforations of the grid 32. The presence of the relatively thin portions can facilitate imaging of the structures within these thin portions of the sample 30.

The planchette-grid-planchette sandwich may then be immersed into, for instance, liquid ethane that is maintained near its melting point with a temperature range of from about −170° C. to about −160° C. Once vitrification of the sample 30 has taken place, the copper planchettes 31 and 33 may be separated (e.g., peeled apart) while they remain immersed in the liquid ethane to remove the grid 32 therebetween. In an embodiment, a cryogenically cooled forceps (not shown) may be used to separate the grid 32 from the two planchettes. Next, the grid 32 may be withdrawn and stored under liquid nitrogen, for instance, on a grid holder, such as that shown in FIG. 1B, until it is ready to be transferred to cold stage 34 for direct imaging of the ultrastructural biomarkers within the sample 30.

This technique can be suited for preparation of highly viscous samples and gels, where blotting may not be feasible, for subsequent high resolution imaging. In other words, those samples that cannot be prepared for imaging using cryo-TEM can be prepared using this M-FFDI technique. This technique can also be employed to prepare samples that have a predominant organic phase that tend to dissolve if exposed directly to ethane. Accordingly, by providing these two approaches for imaging, a substantially complete range of solutions or biological fluids that can be imaged.

The combination of cryogenic vitrification for sample preparation and the high contrast microscopy for imaging of the sample can produce reliable, substantially artifact-free direct images of ultrastructural biomarkers, for instance, nanoscale aggregates in solution, or of soft tissue sections in their native states. It should be appreciated that neither cryo-TEM nor M-FFDI requires the use heavy metal salts to create contrast, thus avoiding salt-related phase transitions. In addition, the structural information obtained from cryo-TEM or M-FFDI, when applied to, for instance, computer based reconstruction of images obtained at different angles or stage tilts, can provide three dimensional (3-D) structural information on macromolecular assemblies. Such 3-D reconstruction is well known in the art, for example, 3-D images created from CAT scans.

Although the use of cryogenic vitrification is described above in connection with cryo-TEM and M-FFDI, it should be appreciated that such can be employed with other imaging approaches. For instance, cryogenic vitrification may be used in connection with Cryotoming to image and examine ultrastructural biomarkers in various tissue samples. As an example, a sample of about 1 mm square may be vitrified by high pressure freezing. The vitrified sample may then be positioned and secured on, for instance, a cold aluminum pin. Thereafter, a section of approximately 50 nm-100 nm may be microtomed (i.e., sliced) using a cold diamond knife. This sample may subsequently be placed on a carbon-coated electron microscope grid, and imaged at from about −170° C. to about −150° C. on a cold stage.

### NSOM Technique:

In certain instances, near-field scanning optical microscopy (NSOM) may be used. In general, NSOM is a photonic instrument having a sub-micron optical probe that can be positioned in proximity to the sample. Light can be transmitted through the tip of the probe to the sample for imaging of the sample.

It should be noted that the near-field is a region above a surface of an object, such as a serum sample in this instance, with dimensions of less than about a single wavelength of the light incident on the surface. Within this region, light is not diffraction limited and can permit nanometer spatial resolution. As such, NSOM can permit non-diffraction limited imaging and spectroscopy of samples.

### DLS Technique:

In addition, if desired, the size of ultrastructural biomarkers in the serum samples can be measured. Size measurements can be taken using, for instance, a dynamic light scattering technique (DLS). DLS can provide quick measurements of mean hydrodynamic diameter of the ultrastructural biomarkers in serum samples. DLS may be performed on the samples using, for instance, Brookhaven Instruments BI-200SM, at a scattering angle of about 90° and a wavelength of about 514.5 nm. The samples may be equilibrated at about 37° C. before measurement to avoid any convection effects. Data may be acquired, for instance, with a BI-9010 AT digital autocorrelator. The diffusion coefficient of the aggregates can be determined using a 4th order cumulant fit to the intensity autocorrelation function. The hydrodynamic size of the aggregates can be calculated using, for instance, the Stokes-Einstein equation.

In DLS, a laser beam passes through the sample. As the objects in the sample move randomly by Brownian motion, they pass in and out of the path of the laser beam. This movement causes a fluctuation in the intensity of the light scattering from the sample. Smaller objects in the sample can cause greater fluctuations in the frequency since smaller objects can be more mobile and can move in and out of the beam path more frequently. This fluctuation in the frequency can be monitored by developing an intensity autocorrelation function, which can be deconvoluted to produce a diffusion coefficient for the objects in the suspension. This diffusion coefficient can be correlated to the size of the objects in the sample and can thus provide measurements for the ultrastructural biomarkers found in the serum.

### EXAMPLES

In accordance with an embodiment of the present invention, ultrastructural biomarkers identified through the use of cryo-TEM, M-FFDI or NSOM may be used for a wide variety of applications, for example, to make early disease screening (i.e., prediction, susceptibility), disease monitoring, early markers of drug related toxicity, and drug efficacy, among others. Other applications that can be contemplated include those for diagnostic, therapeutic, prophylactic, drug discovery, and patient stratification purposes.

A biomarker or biological marker is defined by the FDA as a characteristic that is objectively measured and evaluated as an indicator of normal biologic or pathogenic processes or pharmacological responses to a therapeutic intervention.

Images obtained from a cryo-TEM sample may be analyzed to determine or obtain the morphologies of the quantitative and qualitative data from the profiles of the ultrastructural biomarkers. In an embodiment, the method of the present invention includes obtaining a sample with sufficient volume for analysis using cryo-TEM from a test subject. The test subject may be a human or animal. Samples from healthy individuals and age matched (50±5 years) patients diagnosed with prostate cancer may be obtained from any available clinical collection. Samples may be stored frozen at −70° C. until used.

In an embodiment, the test sample to be used for analysis and subsequent determination of presence or absence of prostate cancer may include a serum sample. The serum sample may include whole serum. Such a sample may be found in a near in situ state. In diseased individuals, the composition of, for instance, serum components can be altered due to cell proliferation, metabolic, hormonal, inflammatory or secretory changes, thus impacting the structure and morphology of the resulting imaged components. As there are numerous diseases where ultrastructural abnormalities occur in cell organelles, tissue structures, and biological fluids, the utilization of ultrastructural analysis of components therein can reveal critical biomarkers associated with these diseases. Moreover, by screening and comparing biomarkers from a sample of a test subject to those biomarkers from a healthy subject or control population for structural or morphological variations, the presence of variations in the ultrastructural biomarkers from the test subject sample, in one embodiment, can act as determinants or predictors of disease, disease predisposition, and disease susceptibility.

Examples of biological fluids from which ultrastructural abnormalities can be observed include, but are not limited to blood, mucosa, plasma, serum, cerebral spinal fluid, spinal fluid, joint fluid, urine, saliva, bile, pancreatic fluid, peritoneal fluid, lung fluid, alveolar sac fluid, sinus fluid, lachrymal fluid, nasal mucous and fluid, intrathoracic fluid, gastric fluid, gastrointestinal fluid, ovarian fluid, testicular, prostrate fluid, uterine fluid, cystic fluid, renal fluid, brain fluid, ophthalmic fluid, tear, ear fluid, auditory canal fluid, subcutaneous or muscular fluid.

Examples of cell organelles and tissue structures within which ultrastructural abnormalities can occur include plasma membrane, organelle membranes, basement membrane, extracellular matrix, intercellular organelles, intercellular structures, intracellular membranes, intracellular organelles, cell-cell junctions, cell-cell adhesion, gap junctions, tight junctions, nucleus, nucleolus, nuclear membrane, nuclear pore, chromosomes, chromatin, ribosomes, polyribosomes, monosomes, cellular proteins, cellular protein complexes, cellular protein subunits, extracellular proteins, extracellular protein complexes, extracellular protein subunits, secretory proteins, secreted protein complexes, secretory protein subunits, secreted intracellular or extracellular protein aggregates, golgi, lysosomes, mitochondria, endosomes, mitochondrial membranes, peroxisomes, endoplasmic reticulum, mRNA, DNA, tRNA, rRNA, small RNA, proteosomes, vacuoles, intracellular and extracellular vesicles, cavity, and droplets, cellular lipids or carbohydrates, cellular lipid or carbohydrate complexes, cellular lipoproteins, cellular glycoproteins, extracellular lipids, extracellular lipoprotein complexes, extracellular lipoprotein subunits, secreted proteins, secreted protein subunits, lipoprotein or glycoprotein aggregates can reveal critical biomarkers associated with these diseases.

In an embodiment, the sample may include one of cellular organelles or components, tissue components, plasma membrane, organelle membranes, basement membrane, extracellular matrix, intercellular organelles, intercellular structures, intracellular membranes, intracellular organelles, cell-cell junctions, cell-cell adhesion, gap junctions, tight junctions, nucleus, nucleolus, nuclear membrane, nuclear pore, chromosomes, chromatin, ribosomes, polyribosomes, monosomes, cellular proteins, cellular protein complexes, cellular protein subunits, extracellular proteins, extracellular protein complexes, extracellular protein subunits, secretory proteins, secreted protein complexes, secretory protein subunits, secreted intracellular or extracellular protein aggregates, golgi, lysosomes, mitochondria, endosomes, mitochondrial membranes, peroxisomes, endoplasmic reticulum, mRNA, DNA, tRNA, rRNA, small RNA, proteosomes, vacuoles, intracellular and extracellular vesicles, cavity, and droplets, cellular lipids or carbohydrates, cellular lipid or carbohydrate complexes, cellular lipoproteins, cellular glycoproteins, intracellular and extracellular lipids, extracellular lipoprotein complexes, extracellular lipoprotein subunits, secreted proteins, secreted protein subunits, lipoprotein or glycoprotein aggregates. In an embodiment, the serum may be treated with lipase, proteinases and immunogold labeled antibodies specific for serum antigens. Treating the serum for antigens may aid in determining the molecular and/or chemical composition of the ultrastructural biomarkers.

Sample preparation for cryo-TEM may be carried out in a controlled environment vitrification system (CEVS). The temperature within the CEVS chamber was maintained at about 37° C. to replicate physiological conditions and the humidity was kept at 100% to avoid sample evaporation artifacts. The frozen serum samples were allowed to thaw quickly at about 25° C. In some instances, the serum samples may be diluted with PBS buffer of about pH 7.4. About 5 μl of the serum sample was withdrawn using a pipette and deposited onto a specially prepared holey carbon grid. The sample was blotted, leaving behind thin films of liquid spanning the grid holes. The grid-bearing sample was then plunged into a liquid ethane reservoir, close to its freezing point. Contact with the cryogen induced rapid solidification of the sample, causing the water in the solution to vitrify rather than crystallize. It should be noted that the Vitrobot automated vitrification system from FEI can alternatively be used.

This rapid vitrification can preserve substantially all of the microstructures in their native hydrated states. The microscope grid was then transferred under positive dry nitrogen pressure to a cold stage (Oxford Instruments Cryotransfer System—CT3500J or Gatan 626DH), and maintained at about −170° C. during phase contrast imaging in the electron microscope (JEOL 2100).

If desired, the size of the ultrastructural biomarkers in the serum sample can be measured using, for instance, DLS, as described above.

In an embodiment, the method of the present invention includes identifying at least one ultrastructural biomarker that can be indicative of prostate cancer within the sample and can serve as a determinant or indicator of the presence or potentially the progression of disease. Ultrastructural biomarkers that can be determinant or indicative of prostate cancer or prostate cancer related diseases can be of various sizes and shapes as shown in FIGS. 4A-E. In an embodiment, these ultrastructural biomarkers include, among others, globules, vesicles, rods, aggregated chains, fur-like aggregates, complex vesicular structures, globules within vesicles, vesicles within vesicles, micelles, discs, and complex structures interacting with globules. Of course, other ultrastructural biomarkers may also be found as the present invention is not intended to be limited in this manner. It should also be noted that spatial positions of proteins within these ultrastructural biomarkers may be included.

Examples of globules and rods that can be illustrative of ultrastructural biomarkers for determining the presence of prostate cancer in a test subject are shown in FIG. 4A. As shown in FIG. 4A, globules 400 can be solid and uniformly round objects. It should be noted that globules 400 may be versatile and prevalent in human serum. In certain instances, globules 400 may be found at normal projection and at a stage tilt of 30°. Rods 402, on the other hand, can be cylindrical elongated objects.

Examples of vesicles and fur-like aggregates that can be illustrative of ultrastructural biomarkers for determining the presence of prostate cancer in a test subject are shown in FIG. 4B. As shown in FIG. 4B, vesicles 404 can be hollow spherical objects with a distinct darker circumference. Vesicles 404, in an embodiment, can be bilayer vesicles or discs. These bilayer vesicles or discs in serum may include different kinds of lipids. Fur-like aggregates are shown as item 406 in FIG. 4B.

Examples of globules within vesicles that can be illustrative of ultrastructural biomarkers for determining the presence of prostate cancer in a test subject are shown in FIG. 4C. As shown in FIG. 4C, a globule within a vesicle 408 may be a vesicle, as described above, situated within a globule, as described above.

Examples of aggregated chains 410 that can be illustrative of ultrastructural biomarkers for determining the presence of prostate cancer in a test subject are shown in FIG. 4D.

Examples of complex vesicular structures 412 that can be illustrative of ultrastructural biomarkers for determining the presence of prostate cancer in a test subject are shown in FIG. 4E.

In an embodiment, the method of the present invention includes obtaining qualitative or quantitative data from the ultrastructural biomarker that can be compared to data from a control subject and may serve as an indicator of the presence, progression or predictor of prostate cancer or prostate related diseases. To determine which ultrastructural biomarkers may be indicative of prostate cancer or prostate related diseases, qualitative and/or quantitative data from the ultrastructural biomarkers can be obtained from a test sample and compared to a sample from a control subject. In an embodiment, qualitative characteristics can be used as a determinant, predictor, or indicator of prostate cancer and its progression. Qualitative characteristics may include one of size, shape, structural morphology, length, width, or combinations thereof. In an embodiment, quantitative characteristics can also be used as a determinant, predictor, or indicator of prostate cancer. Quantitative characteristics, on the other hand, may include one of concentration, absolute number, ratio between one or more ultrastructural biomarkers, or combinations thereof. A comparison of qualitative or quantitative characteristics of two or more ultrastructural biomarkers may also be used as an indicator of prostate cancer or prostate related diseases. Qualitative or quantitative data of the spatial positions of proteins within the ultrastructural biomarkers may also be analyzed as an indicator of prostate cancer or prostate related diseases. It should be appreciated that other qualitative and quantitative characteristics are possible as the present invention is not intended to be limited in this manner. The qualitative or qualitative gathered from the test sample and/or the control sample can be stored for use in future comparisons against test samples to determine, predict or monitor the test sample for prostate cancer or prostate related diseases.

In accordance with another embodiment, the method of the present invention includes comparing the biomarker from the test subject to a substantially similar biomarker in a sample from a control subject for quantitative or qualitative variations in the data, wherein the variations in the data act as a determinant for prostate cancer or prostate related diseases. In an embodiment, a reduction of about ten percent or more in size of the globules can be a determinant of quantitative or qualitative variations, and potentially the presence of prostate cancer. A comparison of globules in healthy samples to globules of prostate cancer samples shows that the size of globules decreases from about 70 nm in the healthy sample to about 30 nm in the prostate cancer sample. This may represent a change ranging from about 20% to about 90% in globule size between a healthy sample and a prostate cancer sample.

It should be noted that a statistically significant reduction in concentration of globules, size of globules, concentration of vesicles, size of vesicles, and in ratio of vesicles to globules can be an indicator of quantitative or qualitative variations, and potentially the presence of prostate cancer. In an embodiment, a statistically significant reduction may include a reduction of from about 1% to at least about 20% or more. In certain instances, a reduction of about 10% to about 20% can be a statistically significant reduction, whereas in other instances, a reduction of about 5% to about 10% or about 2% to about 5% can be statistically significant. In some instances, a reduction of about 1% can be statistically significant.

In accordance with an embodiment of the present invention, ultrastructural biomarkers identified through the use of cryo-TEM, M-FFDI or NSOM can also be used for screening, monitoring, predicting or assessing susceptibility to prostate cancer or prostate related diseases. Similarly, ultrastructural biomarkers identified using a method of the present invention can further be used for evaluating or predicting therapeutic efficacy or response in treatment of prostate cancer or prostate cancer related diseases.

In these embodiments, a first sample and a second sample can be obtained from a subject at different points in time. Using, for instance, the first sample, the method of the present invention includes identifying at least one ultrastructural biomarker that can be indicative of prostate cancer, and thereafter obtaining qualitative or quantitative data from that ultrastructural biomarker.

Once qualitative or quantitative data from an ultrastructural biomarker in the first sample have been obtained, such data in the first sample can be compared to a control sample for qualitative or quantitative variations. The presence of qualitative or quantitative variations can act as a determinant for prostate cancer or prostate related diseases within the first sample. In one embodiment, the qualitative or quantitative data from the first sample can then be compared to that observed in the second sample to obtain qualitative or quantitative variations that can act as a predictor or indicator of susceptibility for prostate cancer or prostate related diseases. Qualitative or quantitative variations can include, as noted above, a reduction in concentration of globules, size of globules, concentration of vesicles, size of vesicles, or size of ultrastructural biomarkers. In this instance, a reduction in concentration of globules, size of globules, concentration of vesicles, size of vesicles, or size of ultrastructural biomarkers can act as a predictor or indicator of susceptibility for prostate cancer or prostate related diseases.

The qualitative and quantitative data from the first sample and second sample can also be compared to one another to obtain qualitative or quantitative variations that can act a marker of therapeutic efficacy in treatment for prostate cancer or prostate cancer diseases. In such an instance, an increase in concentration of globules, size of globules, concentration of vesicles, size of vesicles, or size of ultrastructural biomarkers can act as a marker of therapeutic efficacy in treatment for prostate cancer or prostate cancer diseases.

Additionally, the method of the present invention can be used for assessing risks of toxicity, adverse events, serious adverse events associated with a drug or therapeutic candidate for treatment of prostate cancer or other prostate related disease. With such an approach, the qualitative and quantitative data from the first sample and second sample can then be compared to one another to obtain qualitative or quantitative variations that can act as a marker for risks for toxicity, adverse events or serious adverse events associated with the drug or therapeutic candidate for the treatment of prostate cancer or prostate cancer diseases. In this case, changes in concentration of globules, size of globules, concentration of vesicles, size of vesicles, or size of ultrastructural biomarkers can act as a marker of therapeutic efficacy in treatment for prostate cancer or prostate cancer diseases.

Furthermore, the method of the present invention can be used for screening a subject for clinical trial for treatment of prostate cancer or other prostate related diseases. In an embodiment, a first sample is obtained from a subject. The subject is then treated with a therapeutic candidate for treatment of prostate cancer or prostate related diseases. Following treatment, a second sample is obtained or extracted from the treated subject. Qualitative or quantitative data obtained from at least one ultrastructural biomarker in the first sample is compared to that in the second sample for variations. The presence of quantitative or qualitative variations between the first sample and the second sample can then be correlated with a marker of therapeutic efficacy in treatment for prostate cancer or prostate cancer diseases and the subject can then be enrolled into a clinical trial for the therapeutic candidate based on the therapeutic efficacy of the therapeutic candidate. In this instance, an increase or reduction in concentration of globules, size of globules, concentration of vesicles, size of vesicles, or size of ultrastructural biomarkers can act as a marker of therapeutic efficacy in treatment for prostate cancer or prostate cancer diseases. It should be appreciated that the method can also include identifying at least one ultrastructural biomarker indicative of prostate cancer within the first sample and second sample and obtaining qualitative or quantitative data from the ultrastructural biomarker in the first sample and second sample.

### Experiment 1

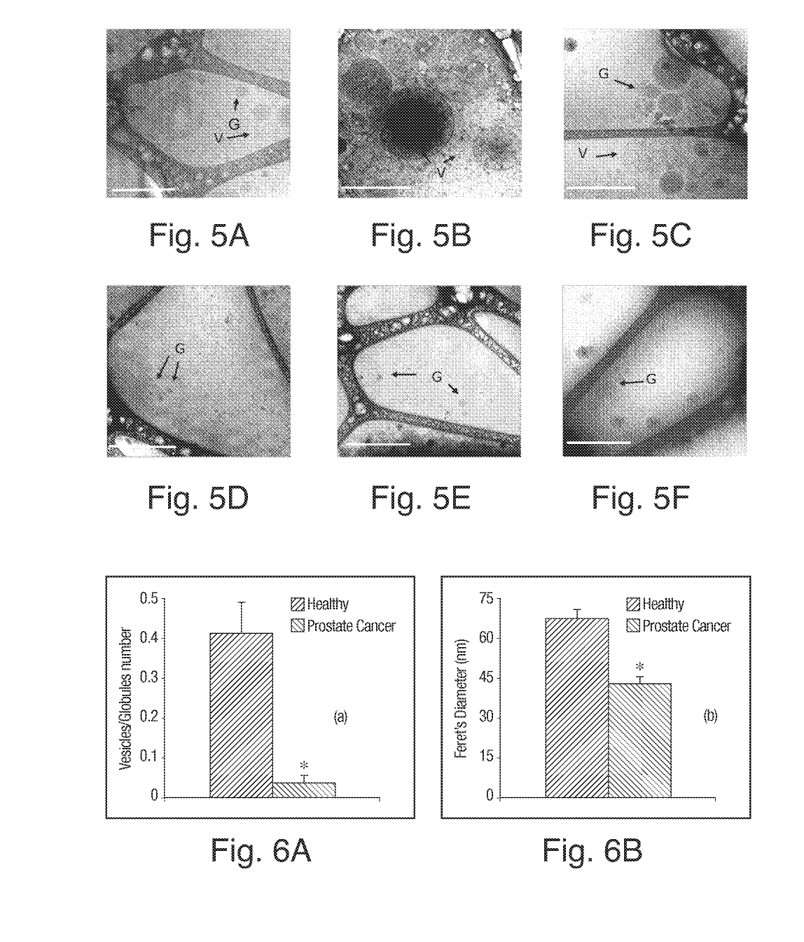

In this experiment, a serum sample from a test subject was prepared in the manner set forth above and subsequently imaged through the use of Cryo-TEM. In FIGS. 5A-F, there is illustrated a comparison of the structural and physical changes between a control sample (FIGS. 5A-C) and a prostate cancer sample (FIGS. 5D-F). In this instance, the prostate cancer samples had PSA levels between 4-10 ng/ml. All the patients were pre-diagnosed with adenocarcinoma of the prostate with Gleason scores of either 6 or 7, and stages varying from 1 to 3.

As shown in FIGS. 5A-F, images included globules (G) and vesicles (V) from serum of healthy subjects and globules (G) and vesicles (V) from serum of prostate cancer subjects. In one instance, images included 1251 globules and 352 vesicles from serum of healthy subjects and 1141 globules and 67 vesicles from serum of prostate cancer subjects. These numbers included serum samples from seven healthy subjects and nine samples from prostate cancer subjects. The size of each identified structure was measured using ImageJ 1.36b image processing software.

Comparison and analysis of the images from serum of healthy subjects and serum of prostate cancer subjects, shown in FIGS. 5A-F, revealed several differences. First, the size of globules can be greater in serum from healthy subjects than in serum of prostate cancer subjects. Second, the relative number of vesicles to globules can be greater in serum from healthy individuals than in serum from individuals with prostate cancer. These differences in morphologies as well as the qualitative and quantitative data represent, in this case, a significant indication for the presence of prostate cancer.

### Experiment 2

In this experiment, a serum sample from a test subject was prepared in the manner set forth above and subsequently imaged through the use of Cryo-TEM. In an embodiment, vesicle-to-globule number ratio may be compared between healthy and prostate cancer subjects. FIG. 6A illustrates a comparison of vesicles to globules in healthy subjects and prostate cancer subjects.

As shown in FIG. 6A, the mean vesicle-to-globule number ratio for healthy and prostate cancer subjects is about 0.39±0.28 and about 0.05±0.03 respectively. The mean for the healthy subject, in this instance, is greater than the mean for the prostate cancer subject (p=0.000087). This finding suggests that a significant reduction in the ratio of vesicles to globules can be an indicator of qualitative or quantitative variations. One possible explanation for the results shown in FIG. 6A is that there may be increased uptake of lipids such as cholesterol by fast growing prostate cancer cells. It has been shown that prostate cancer cells lack the sterol feedback regulation of normal prostate cells due to the overproduction of low density lipoprotein receptor (LDLR). It has also been shown that addition of exogenous cholesterol does not down-regulate LDLR and one of the regulator of LDLR promoter (SREBP2) in prostate cancer cells unlike the normal prostate cells. This may suggest that due to an increased uptake of cholesterol lipids by prostate cancer cells, less number of free lipid vesicles should be present in the serum of prostate cancer subjects.

### Experiment 3

Here, a serum sample from a test subject was prepared in the manner set forth above and subsequently imaged through the use of Cryo-TEM. In an embodiment, size of the globules may be compared for healthy and prostate cancer subjects. In FIG. 6B illustrates a comparison of globule size in health subjects and prostate cancer subjects.

As shown in FIG. 6B, the mean sizes of globules from healthy and prostate cancer subjects are about 57.8±13.7 nm and about 29.5±9.2 nm respectively. It should be noted that these numbers represent a spread of about 30% and a statistically significant decrease (p=0.00035) in the mean globule size in the serum sample of a prostate cancer subject as compared to the serum sample of a healthy subject. Results from FIG. 6B may suggest that a reduction of about twenty percent or more in size of globules may be an indicator of qualitative or quantitative variations.

A similar serum sample from a prostate cancer subject was compared to a sample from a healthy subject. The results show that the size of the globules decreases from about 70 nm in healthy subjects to about 30 nm in prostate cancer subjects. This represents a change ranging from about 20% to about 90% in globule size between healthy subjects and prostate cancer subjects.

### Experiment 4

In this experiment, dynamic light scattering (DLS) was used to measure the size of the ultrastructural biomarkers found in a serum sample from a healthy subject and a serum sample from a prostate cancer subject.

Results obtained using DLS analysis revealed that the mean sizes of ultrastructural biomarkers in serum of healthy and prostate cancer subjects were about 241.0±128.0 nm and about 44.7±23.8 nm respectively (p=0.00058). These results may be consistent with the results obtained through cryo-TEM experiments in that the sizes of the ultrastructural biomarkers decreases with the presence of prostate cancer. As the DLS results suggest, a significant reduction in the size of ultrastructural biomarkers can be a determinant or indicator of qualitative or quantitative variations, or the presence of prostate cancer. It should be noted that the sizes produced by DLS correspond to all the different ultrastructural biomarkers in the serum and not only to globules. In addition, larger particles that may not be accommodated into the thin film required for Cryo-TEM analysis may be included in the DLS analysis. By including larger particles in the analysis, the sizes yielded by the DLS analysis may be greater than by the Cryo-TEM analysis.

### Mouse Model of Human Prostate Cancer

In an embodiment, Cryo-TEM imaging may be conducted using a murine model of human prostate cancer in Nod-Scid mice by orthotopic transplantation. A serum sample from a mouse was prepared in the manner set forth above and subsequently imaged through the use of Cryo-TEM. Characteristics of the mouse samples were obtained using an automated vitrification system, for instance, the Vitrobot system from FEI. Once the sample is vitrified, it is transferred onto a cold stage, inserted into the electron microscope and imaged. After an image is obtained, image analysis software called ImageJ (from NIH) was used to clearly delineate, or mark manually, the ultrastructural biomarkers of interest. The software provides the mean size and the number concentration from each ultrastructural biomarker. The numbers may then be averaged over multiple images to obtain the mean values of size and concentration for ultrastructural biomarker of interest.

### Experiment 5

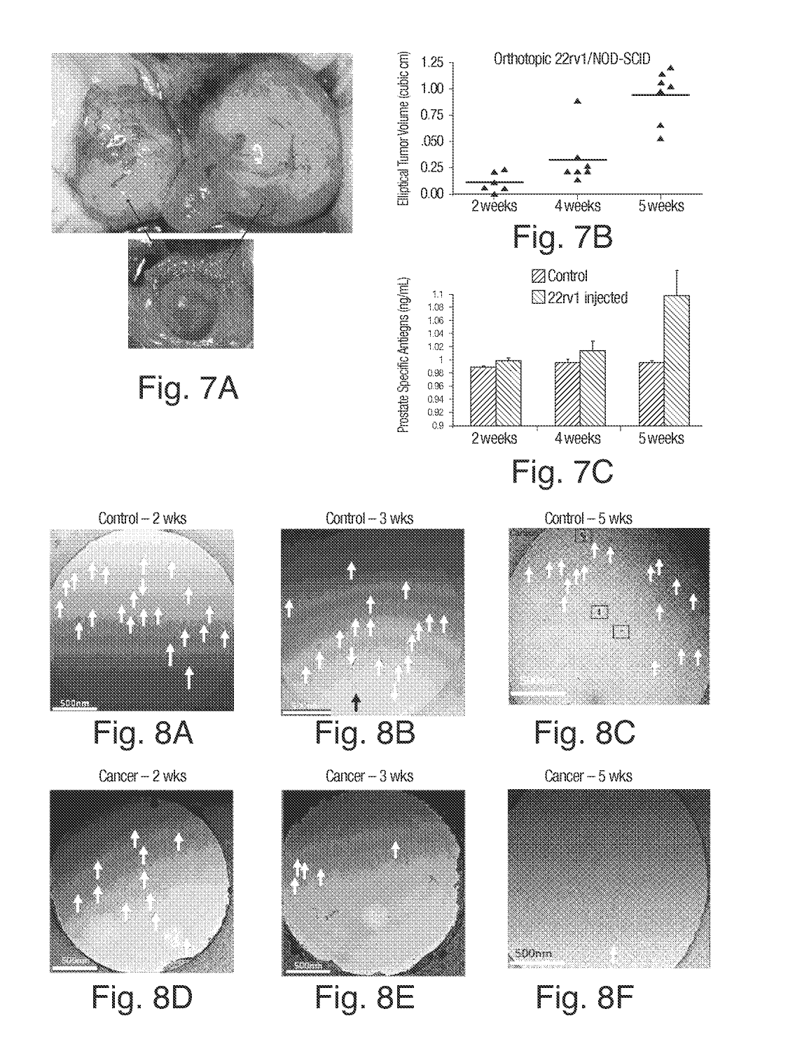

Here, a serum sample from a test subject was prepared in the manner set forth above and subsequently imaged through the use of Cryo-TEM. In an embodiment, cryo-TEM imaging of prostate tumor growth and serum PSA levels over 2, 3 and 5 weeks in a murine model may be analyzed. FIGS. 7A-C illustrate prostate tumor growth and serum PSA levels in tumor-bearing prostate. The prostate tumor volume of these mice was measured and photographed after sacrifice. In addition, these samples may be collected and stored at −80 C for cryo-TEM processing and imaging.

As shown in FIGS. 7A-C, there can be a progressive increase in tumor growth. FIG. 7A shows an enlarged prostate within the abdominal cavity. FIG. 7B illustrates enlargement of the volume of the tumor measured over week 2, week 4 and week 5 whereas FIG. 7C illustrates an increase in serum PSA levels in the tumor bearing mice over the course of the tumor growth. These findings suggest that the tumor continuously grows over at least a five week time span and may be becoming increasing malignant over time.

### Experiment 6

In this experiment, a serum sample from a test subject was prepared in the manner set forth above and subsequently imaged through the use of Cryo-TEM. In an embodiment, cryo-TEM images of serum from any number of mice from each time-course group of tumor growth (2, 3 and 5 weeks from control and tumor bearing mice) in a murine model of human prostate cancer may be analyzed. FIGS. 8A-F illustrate Cryo-TEM images of control and prostate cancer mice at 2 weeks, 3 weeks and 5 weeks. FIGS. 8A-C illustrate Cryo-TEM images of control mice at 2 weeks, 3 weeks and 5 weeks and FIGS. 8D-F illustrate Cryo-TEM images of prostate cancer mice at 2 weeks, 3 weeks and 5 weeks.

As shown in FIGS. 8A-F, there can be a progressive decrease in the concentration of globules in the images derived from late stages of tumor growth. For example, in the serum of 2-week cancer bearing mice, there can be a reduction of globules as compared to a control sample. This decrease in globules can be progressive over time and by 3 weeks the change between the prostate cancer sample and the control sample may be more evident. As shown in FIG. 8F, at 5 weeks of tumor growth, the reduction in globule number may be significant in that no more than about 1 to about 3 globules can be seen per image in all of mice sera that were analyzed. This reduction in globule number serves as a structural marker within the serum for the progression of the tumor growth. The ability of cryo-TEM images to detect early changes in structural markers within the serum of tumor bearing mice can be evidence that the method of the present invention has the potential to give early read-outs of prostate cancer in clinical applications.

### Other Applications

Moreover, it is well established that structural and morphological variations in secreted components of biological fluids, such as serum, precede or occur simultaneously with functional changes. Accordingly, the ability to monitor the structural or morphological changes in aggregates present in biological fluids in their native, hydrated states at nanoscale resolution, and which can be correlated to functional and phenotypic changes has the potential for early and simple detection of disease, classification of disease sub-categories, and monitoring of disease progression. In one embodiment, to effectively monitor the progress of a disease via an image-based platform, such as that employed in the present invention, an accurate, precise and temporally contiguous picture of the progress of the disease is needed. The method of the present invention can provide an accurate and precise image of the ultrastructural biomarkers from samples taken from a subject over a period of time. As a result, these images may be compared against one another for any structural or morphological changes in the biomarkers being observed to determine and monitor the progression of the disease.

The ultrastructural biomarkers identified can also be employed for drug or biological therapeutics screening, monitoring, predicting or assessing susceptibility. For example, in cell-based or in vitro drug screening, any intracellular or extracellular markers of change can be detected and utilized as a marker of drug or therapeutic efficacy or an indicator that the drug target is being hit. In particular, in one embodiment, different drugs, candidate drugs or therapeutics may be administered to test subjects, and the side effects, including desired effects, toxicity, adverse effects or serious adverse effects, may be documented. Any conventional metrics of side effect severity can be used. For instance, a reduction in concentration of globules, size of globules, concentration of vesicles, size of vesicles, or size of ultrastructural biomarkers from a first sample taken from a subject to a second and subsequent sample taken from that subject can act as a predictor or indicator of susceptibility for prostate cancer or prostate related diseases. On the other hand, an increase in concentration of globules, size of globules, concentration of vesicles, size of vesicles, or size of ultrastructural biomarkers from a first sample taken from a subject to a second and subsequent sample taken from that subject can act as a marker of therapeutic efficacy in treatment for prostate cancer or prostate cancer diseases. In addition, before and after drug administration, the biomarkers may be identified and analyzed to determine which of the biomarkers has changed. In this way, the biomarkers affected by each drug can be correlated with the particular desirable and undesirable effects of the drug.

It is anticipated that new drugs being developed will have fewer adverse effects due to extensive use of biomarkers to identify adverse events in preclinical animal models or in clinical trial patients. As additional generations of drugs continues to be developed, the list of relevant biomarkers and their change can be refined further. In addition, as it becomes clear whether each biomarker is indicative of desired or undesired effects, more information about the mechanisms of drug action are learned, helping to direct development of next generation drugs. Accordingly, these ultrastructural biomarkers can allow for the monitoring and evaluating of drug or therapeutic safety and efficacy during discovery, preclinical and all levels of clinical trials, as well as post sales monitoring and testing. Furthermore, a similar approach may be used to determine and evaluate patient response or response rate, as well as clinical trial participant response or response rate.

Similarly, the above protocol can be employed to generate information that can lead to the understanding of the risks of adverse events, toxicity or serious adverse events associated with marketed drugs or therapeutics, drug or therapeutic candidates, as well as risks for drug attrition. Such an understanding can assist in a decision making process during clinical development, thereby driving informed stop/go decisions early in, or prior to clinical development. The information may also be used, in an embodiment, in designing and developing drugs or therapeutics that can be tailored to address only relevant disease mechanisms while causing fewer adverse effects.

The present invention, in addition to being able to resolve ultra-structural features in the morphology of cells, cellular organelles, and extracellular matrix, can also employ Cryo-TEM, M-FFDI or NSOM to detect macromolecular structural differences in lipid droplets, vesicles, and other structural components in biological fluids.

Furthermore, the present invention permits, in an embodiment, identification of the spatial positions of proteins in a larger assembly or changes of protein complex morphology. This is important because proper assembly can be critical to the functioning of the protein complexes and cell organelles. In particular, there are potential changes in morphology and aggregates in different stages of disease which can change size or size distribution. Since these changes are physical, the identification process employed by a method of the present invention does not require any a priori knowledge of specific biological targets. Accordingly, sole reliance on biochemical assays can be eliminated.

The high resolution images of these ultrastructures, ranging from nanometers to micrometers in size, thus provide clear indications that cryogenic vitrification and high contrast imaging achieved through cryo-TEM, M-FFDI or NSOM can generate a powerful tool for analyzing these nanostructures and changes to these nanostructures in biological samples. Whether, the sample is fluid or viscous, the provision of the either cryo-TEM, M-FFDI or NSOM, as disclosed herein, can create broad capability to examine relevant bio-samples under conditions that most closely resemble their native states. For instance, biomarkers that are from any of parts of the human body, including any viruses, bacteria or other pathogens residing in any part of the human body can be identified employing the methods of the present invention.

Moreover, since the present invention involves utilization of resolutions relatively far beyond those traditionally used, the potential for discovery of early changes of structural markers can be substantially high. Thus, the use of cryo-TEM, M-FFDI or NSOM coupled with image analysis can provide a novel and high resolution approach for the identification of ultrastructural biomarkers. Furthermore, such an approach has the potential to change the paradigm and dramatically reduce the cost associated with biomarker discovery and validation, by providing a robust and relatively sensitive approach to diagnosing and monitoring diseases, while simultaneously reducing drug development costs.

Although the above description has been provided in the context of human subjects, it can be equally well applied to animal models. For instance, suitable animals include mice, rats, and rabbits.

While the invention has been described in connection with the specific embodiments thereof, it will be understood that it is capable of further modification. Furthermore, this application is intended to cover any variations, uses, or adaptations of the invention, including such departures from the present disclosure as come within known or customary practice in the art to which the invention pertains, and as fall within the scope of the appended claims.

What is claimed is:

1. A method for determining presence of prostate cancer or prostate related diseases, the method comprising:
obtaining a test sample from a test subject;
identifying at least one ultrastructural biomarker indicative of prostate cancer within the sample;
obtaining qualitative or quantitative data from the ultrastructural biomarker in the test sample; and
comparing the qualitative or quantitative data from the biomarker in the sample from the test subject to that in a sample from a control subject for variations, wherein the presence of qualitative or quantitative variations can act as a determinant for prostate cancer or prostate related diseases.

2. A method as set forth in claim 1, wherein the step of obtaining includes obtaining a sample with sufficient volume for analysis.

3. A method as set forth in claim 1, wherein, in the step of obtaining, the sample includes serum.

4. A method as set forth in claim 1, wherein, in the step of obtaining, the sample includes one of cellular organelles or components, tissue components, plasma membrane, organelle membranes, basement membrane, extracellular matrix, intercellular organelles, intercellular structures, intracellular membranes, intracellular organelles, cell-cell junctions, cell-cell adhesion, gap junctions, tight junctions, nucleus, nucleolus, nuclear membrane, nuclear pore, chromosomes, chromatin, ribosomes, polyribosomes, monosomes, cellular proteins, cellular protein complexes, cellular protein subunits, extracellular proteins, extracellular protein complexes, extracellular protein subunits, secretory proteins, secreted protein complexes, secretory protein subunits, secreted intracellular or extracellular protein aggregates, golgi, lysosomes, mitochondria, endosomes, mitochondrial membranes, peroxisomes, endoplasmic reticulum, mRNA, DNA, tRNA, rRNA, small RNA, proteosomes, vacuoles, intracellular and extracellular vesicles, cavity, and droplets, cellular lipids or carbohydrates, cellular lipid or carbohydrate complexes, cellular lipoproteins, cellular glycoproteins, intracellular and extracellular lipids, extracellular lipoprotein complexes, extracellular lipoprotein subunits, secreted proteins, secreted protein subunits, lipoprotein or glycoprotein aggregates.

5. A method as set forth in claim 1, wherein, in the step of identifying, the ultrastructural biomarker indicative of prostate cancer includes one of vesicles, globules, rods, aggregated chains, fur-like aggregates, complex vesicular structures, globules within vesicles, vesicles within vesicles, or other similar ultrastructural biomarkers.

6. A method as set forth in claim 1, wherein, in the step of measuring, the qualitative data includes one of size, shape, structural morphology, length, width, or a combination thereof.

7. A method as set forth in claim 1, wherein, in the step of obtaining, the quantitative data includes one of concentration, absolute number, ratio, or a combination thereof.

8. A method as set forth in claim 1, wherein, in the step of comparing, the test subject and control subject includes human subjects or animal subjects.

9. A method as set forth in claim 1, wherein, in the step of comparing, a reduction of about twenty percent or more in size of globules can act as a determinant for prostate cancer or prostate related diseases.

10. A method as set forth in claim 1, wherein, in the step of comparing, a statistically significant reduction in concentration of globules can act as a determinant for prostate cancer or prostate related diseases.

11. A method as set forth in claim 1, wherein, in the step of comparing, a statistically significant reduction in ratio of vesicles to globules can act as a determinant for prostate cancer or prostate related diseases.

12. A method as set forth in claim 1, wherein, in the step of comparing, a statistically significant reduction in concentration of vesicles can act as a determinant for prostate cancer or prostate related diseases.

13. A method as set forth in claim 1, wherein, in the step of comparing, a statistically significant reduction in size of vesicles can act as a determinant for prostate cancer or prostate related diseases.

14. A method as set forth in claim 1, wherein, in the step of comparing, a statistically significant reduction in size of ultrastructural biomarkers can act as a determinant for prostate cancer or prostate related diseases.

15. A method for screening, monitoring, predicting or assessing susceptibility to prostate cancer or prostate related diseases, the method comprising:
obtaining from a subject a first sample and a second sample taken at different points in time;
identifying in the first sample at least one ultrastructural biomarker indicative of prostate cancer;
obtaining qualitative or quantitative data from the ultrastructural biomarker in the first sample;
comparing the qualitative or quantitative data of the biomarker in the first sample to that in the second sample for variations; and
associating the presence of the qualitative or quantitative variations between the first sample and the second sample as a predictor or indicator of susceptibility for prostate cancer or prostate related diseases.

16. A system as set forth in claim 15, wherein, in the step of obtaining, the sample includes serum.

17. A method as set forth in claim 15, wherein, in the step of identifying, the ultrastructural prostate cancer biomarker includes one of vesicles, globules, rods, aggregated chains, fur-like aggregates, complex vesicular structures, globules within vesicles, vesicles within vesicles, or other similar ultrastructural biomarkers.

18. A method as set forth in claim 15, wherein, in the step of obtaining, the qualitative characteristic includes one of size, shape, structural morphology, length, width, or a combination thereof.

19. A method as set forth in claim 15, wherein, in the step of obtaining, the quantitative characteristic includes one of concentration, sheer number, ratio, or a combination thereof.

20. A method as set forth in claim 15, wherein the step of obtaining further includes comparing the qualitative or quantitative data from the ultrastructural biomarker in the first sample to a control sample for qualitative or quantitative variations, wherein the presence of qualitative or quantitative variations can act as a determinant for prostate cancer or prostate related diseases.

21. A method as set forth in claim 15, wherein, in the step of associating, the qualitative or quantitative variations include a reduction in concentration of globules, size of globules, concentration of vesicles, size of vesicles, or size of ultrastructural biomarkers.

22. A method as set forth in claim 15, wherein, in the step of associating, a reduction in concentration of globules, size of globules, concentration of vesicles, size of vesicles, or size of ultrastructural biomarkers can act as a predictor or indicator of susceptibility for prostate cancer or prostate related diseases.

23. A method for evaluating or predicting therapeutic efficacy or response in treatment of prostate cancer or prostate cancer related diseases, the method comprising:
obtaining from a subject a first sample and a second sample taken at different points in time;
identifying in the first sample at least one ultrastructural biomarker indicative of prostate cancer;
obtaining qualitative or quantitative data from the ultrastructural biomarker in the first sample;
comparing the qualitative or quantitative data of the biomarker in the first sample to that in the second sample for variations; and
equating the presence of quantitative or qualitative variations between the first sample and the second sample as a marker of therapeutic efficacy in treatment for prostate cancer or prostate cancer diseases.

24. A system as set forth in claim 23, wherein, in the step of obtaining, the sample includes serum.

25. A method as set forth in claim 23, wherein, in the step of identifying, the ultrastructural prostate cancer biomarker includes one of vesicles, globules, rods, aggregated chains, fur-like aggregates, complex vesicular structures, globules within vesicles, vesicles within vesicles, or other ultrastructural biomarkers.

26. A method as set forth in claim 23, wherein the step of obtaining further includes comparing the qualitative or quantitative data from the ultrastructural biomarker in the first sample to a control sample for qualitative or quantitative variations, wherein the presence of qualitative or quantitative variations can act as a determinant for prostate cancer or prostate related diseases.

27. A method as set forth in claim 23, wherein the step of equating includes associating or correlating the quantitative or qualitative variations as a marker of response rate in clinical trial participants, patients, or in animal models.

28. A method as set forth in claim 23, wherein, in the step of equating, the qualitative and quantitative variations include an increase in concentration of globules, size of globules, concentration of vesicles, size of vesicles, or size of ultrastructural biomarkers.

29. A method as set forth in claim 23, wherein, in the step of equating, an increase in concentration of globules, size of globules, concentration of vesicles, size of vesicles, or size of ultrastructural biomarkers can act as a marker of therapeutic efficacy in treatment for prostate cancer or prostate cancer diseases.

30. A method for assessing risks of toxicity, adverse events, serious adverse events associated with a drug or therapeutic candidate for treatment of prostate cancer or other prostate related disease, the method comprising:
obtaining from a subject a first sample and a second sample taken at different points in time;
identifying in the first sample ultrastructural biomarker indicative of prostate cancer;
obtaining qualitative or quantitative data from the ultrastructural biomarker in the first sample;
comparing the qualitative or quantitative data of the biomarker in the first sample to that in a second sample for variations; and
correlating the presence of qualitative or quantitative variations as a marker for risks for toxicity, adverse events or serious adverse events associated with the drug or therapeutic candidate for the treatment of prostate cancer or prostate cancer diseases.

31. A system as set forth in claim 30, wherein, in the step of obtaining, the sample includes serum.

32. A method as set forth in claim 30, wherein, in the step of identifying, the ultrastructural prostate cancer biomarker includes one of vesicles, globules, rods, aggregated chains, fur-like aggregates, complex vesicular structures, globules within vesicles, and vesicles within vesicles.

33. A method as set forth in claim 30, wherein, in the step of comparing, the subject or control population includes human subjects or animal subject.

34. A method as set forth in claim 30, wherein, in the step of correlating, the qualitative and quantitative variations include an increase or reduction in concentration of globules, size of globules, concentration of vesicles, size of vesicles, or size of ultrastructural biomarkers.

35. A method as set forth in claim 30, wherein, in the step of correlating, an increase or reduction in concentration of globules, size of globules, concentration of vesicles, size of vesicles, or size of ultrastructural biomarkers can act as a marker of therapeutic efficacy in treatment for prostate cancer or prostate cancer diseases.

36. A method for screening a subject for clinical trial for treatment of prostate cancer or other prostate related diseases, the method comprising:
obtaining a first sample from a subject;
treating the subject with a therapeutic candidate for treatment of prostate cancer or prostate related diseases;
extracting a second sample from the treated subject;
comparing qualitative or quantitative data obtained from at least one ultrastructural biomarker in the first sample to that in the second sample for variations;
correlating the presence of quantitative or qualitative variations between the first sample and the second sample as a marker of therapeutic efficacy in treatment for prostate cancer or prostate cancer diseases; and
enrolling the subject into a clinical trial for the therapeutic candidate based on the therapeutic efficacy of the therapeutic candidate.

37. A method as set forth in claim 36, wherein the method further includes identifying at least one ultrastructural biomarker indicative of prostate cancer within the first sample and second sample.

38. A method as set forth in claim 36, wherein the method further includes obtaining qualitative or quantitative data from the ultrastructural biomarker in the first sample and second sample.

39. A method as set forth in claim 36, wherein, in the step of enrolling, an increase or reduction in concentration of globules, size of globules, concentration of vesicles, size of vesicles, or size of ultrastructural biomarkers can act as a marker of therapeutic efficacy in treatment for prostate cancer or prostate cancer diseases.
